(19) United States
(12) Patent Application Publication
(10) Pub. No.: US 2008/0287823 A1
(43) Pub. Date: Nov. 20, 2008

(54) Index Determination

(76) Inventor: Scott Matthew Chetham, Del Mar, CA (US)

Correspondence Address: SHAY GLENN LLP, 2755 CAMPUS DRIVE, SUITE 210, SAN MATEO, CA, 94403, US

(21) Appl. No.: 11/996,065

(22) PCT Filed: Jul. 19, 2006
(86) PCT No.: PCT/AU2006/001022

§ 371 Date: Jul. 25, 2008

Related U.S. Application Data
(60) Provisional application No. 60/703,324, filed on Jul. 28, 2005.

(30) Foreign Application Priority Data
Jul. 20, 2005 (AU) 2005903886

(51) Int. Cl. A61B 5/053

(52) U.S. Cl. 600/547

(57) ABSTRACT

A method of determining an index indicative of the presence, absence or degree of left ventricular hypertrophy in a subject. The method includes using a processing system to determine a measured impedance value for at least one body segment. For each body segment the measured impedance values are used to determine at least one impedance parameter, which are then used to determine a fat-free mass for the subject. The fat free mass can then be used as the index.

### BACKGROUND OF THE INVENTION

The present invention relates to a method and apparatus for monitoring biological parameters, and in particular to a method and apparatus for performing impedance measurements for indexing left ventricular mass.

1. Description of the Prior Art

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

The clinical management of heart failure consumes approximately 1% to 2% of the health care budget in developed countries, with the majority of this expense due to costs associated with hospitalisation. A pan-European survey has shown that up to 65% of patients who are hospitalised for clinical heart failure have had previous admissions for such a condition. Typically admission for clinical heart failure lasts for an average of 11 days with a risk of re-hospitalisation risk of 24%.

Left ventricular hypertrophy (LVH) is a particular heart condition in which the cardiac muscle becomes enlarged with the fibres of the heart muscle becoming thickened and shortened and consequently less able to relax. In general a ventricle wall thickness of greater than about 1.5 cm is considered enlarged and indicative of LVH. LVH typically occurs due to an increased resistance in circulation and may therefore result from a number of different causes, such as hypertension, overexercise, or the like. Whilst LVH can typically be treated through the use of appropriate drugs, surgery, or appropriate lifestyle changes, its diagnosis can prove difficult.

Currently, diagnostic techniques generally use echocardiography or magnetic resonance imaging (MRI) or Spiral CT scanning.

In the case of echocardiography, the patient's heart is imaged using ultrasound, with the images being used to determine left ventricular end-diastolic diameter, the interventricular septum thickness and the posterior wall thickness, which are then, in turn used to derive the left ventricular mass (LVM). The LVM is then used as an indicator of the presence of LVH.

It has been shown that Left Ventricular Mass in normal healthy subjects is correlated to the amount of Fat Free Mass of an individual. A particular problem is regardless of the measurement technique used to find left ventricular mass it requires indexing to obtain a measurement which is clinically useful in people. The current gold standard of DEXA (Dual Energy X-ray Absortiometry) is used to determine Fat Free Mass. In the case of DEXA, this involves X-ray absorption scanning which is used to determine the patient's fat-free mass, which is in turn used as an indicator of the patient's LVM.

However, DEXA scanning can only be performed in limited circumstances due to limited equipment availability and the requirement of the apparatus that a scanning arm move over the patient, which limits the size of patient on which this technique can be used.

Accordingly, there is a need for an alternative mechanism for determining the Fat Free Mass in order to index Left Ventricular Mass.

### SUMMARY OF THE PRESENT INVENTION

In a first broad form the present invention provides a method of determining an index indicative of the presence, absence or degree of left ventricular hypertrophy in a subject, the method including, in a processing system:
a) determining a measured impedance value for at least one body segment;
b) for each body segment, and using the measured impedance values, determining at least one impedance parameter;
c) using each determined impedance value to determine a fat-free mass for the subject; and,
d) determining the index at least in part using the fat-free mass.

Typically the method includes, in the processing system determining the index using the fat-free mass and an indication of a measured left ventricular mass.

Typically the method includes, in the processing system:
a) comparing the index to a reference; and,
b) determining the presence, absence or degree of LVH using the results of the comparison.

Typically the reference includes at least one of:
a) a predetermined threshold;
b) a tolerance determined from a normal population;
c) a predetermined range; and,
d) an index previously determined for the subject.

Typically the method includes, in the processing system, displaying at least one of:
a) a fat free mass;
b) a determined index;
c) a ventricular mass;
d) normal ranges for the index; and,
e) normal ranges for fat free mass; and,
f) normal ranges left ventricular mass.

Typically the method includes determining the ranges in accordance with subject parameters.

Typically the method includes, in the processing system:
a) determining a plurality of measured impedance values for each body segment, each measured impedance value being measured at a corresponding measurement frequency; and,
b) determining the impedance parameters based on the plurality of measured impedance values.

Typically the parameter values include R₀ and R_{∞}, wherein:
i) R₀ is the resistance at zero frequency; and,
ii) R_{∞} is the resistance at infinite frequency.

Typically the method includes, in the processing system, determining the parameter values using the equation:

$$i ) Z = {R}_{∞} + \frac{{R}_{0} - {R}_{∞}}{1 + {( jωτ )}^{( 1 - α )}}$$

ii) where:
   (a) Z is the measured impedance at angular frequency ω,
   (b) τ is a time constant, and
   (c) α has a value between 0 and 1.

Typically the method includes, in the processing system:
a) determining the impedance of each body segment at four discrete frequencies; and,
b) determining values for the parameters by solving the equation using four simultaneous equations.

Typically the method includes, in the processing system, determining the parameter values by:
a) determining an impedance locus using the measured impedance values; and,
b) using the impedance locus to determine the parameter values.

Typically the method includes, in the processing system:
a) causing one or more electrical signals to be applied to the subject using a first set of electrodes, the one or more electrical signals having a plurality of frequencies;
b) determining an indication of electrical signals measured across a second set of electrodes applied to the subject in response to the applied one or more signals;
c) determining from the indication and the one or more applied signals, an instantaneous impedance value at each of the plurality of frequencies; and,
d) determining the index using the instantaneous impedance values.

Typically the method includes, in the processing system:
a) determining at least one impedance measurement to be performed;
b) determining at least one electrode arrangement associated with the determined impedance measurement;
c) displaying a representation indicative of the electrode arrangement; and,
d) causing the impedance measurement to be performed once the electrodes have been arranged in accordance with the displayed representation.

Typically the method includes, in the computer system, displaying an indication of at least one of:
a) the parameter values;
b) the fat-free mass; and,
c) an indication of the at least one of the presence, absence or degree of LVH.

In a second broad form the present invention provides apparatus for determining an index indicative of the presence, absence or degree of left ventricular hypertrophy in a subject, the apparatus includes a processing system for:
a) determining a measured impedance value for at least one body segment;
b) for each body segment, and using the measured impedance values, determining at least one impedance parameter; and,
c) using each determined impedance value to determine a fat-free mass for the subject; and,
d) determining the index at least in part using the fat-free mass.

Typically the apparatus includes:
a) a current supply for generating an alternating current at each of a plurality of frequencies;
b) at least two supply electrodes for applying the generated alternating current to a subject;
c) at least two measurement electrodes for detecting a voltage across the subject; and,
d) a sensor coupled to the measurement electrodes for determining the voltage, the sensor being coupled to the processing system to thereby allow the processing system to determine the measured impedances.

Typically the processing system is for performing the method of the first broad form of the invention.

In a third broad form the present invention provides a method of diagnosing the presence, absence or degree of left ventricular hypertrophy in a subject, the method including, in a processing system:
a) determining a measured impedance value for at least one body segment;
b) for each body segment, and using the measured impedance values, determining at least one impedance parameter;
c) using each determined impedance value to determine a fat-free mass for the subject; and,
d) determining an index at least in part using the fat-free mass, the index being indicative of the presence, absence or degree of left ventricular hypertrophy.

It will be appreciated that the broad forms of the invention may be used individually or in combination, and may be used for diagnosis of the presence, absence or degree of left ventricular hypertrophy in subjects such as humans.

### BRIEF DESCRIPTION OF THE DRAWINGS

An example of the present invention will now be described with reference to the accompanying drawings, in which:

FIG. 1 is a schematic diagram of an example of impedance determination apparatus for providing an index of Left Ventricular Mass;

FIG. 2 is a flowchart of an example of a process for performing impedance determination;

FIG. 3 is a schematic diagram of a second example impedance determination apparatus for providing an index of Left Ventricular Mass;

FIG. 4 is a flowchart of an example of a process for indexing Left Ventricular Mass;

FIGS. 5A and 5B are a flow chart of a first specific example of a process for providing an index of Left Ventricular Mass;

FIGS. 6A to 6D are schematic examples of electrode arrangements for use in the process of FIGS. 5A and 5B;

FIG. 7 is a flow chart of an example of a process for placing the electrodes in the process of FIGS. 5A and 5B;

FIG. 8 is a schematic diagram of a third example of apparatus for providing an index of Left Ventricular Mass;

FIG. 9 is a schematic of an example of an equivalence circuit for modelling a subject's impedance response;

FIG. 10 is an example of a “Wessel” plot of a subject's impedance response.

### DETAILED DESCRIPTION OF THE PREFERRED EMBODIMENTS

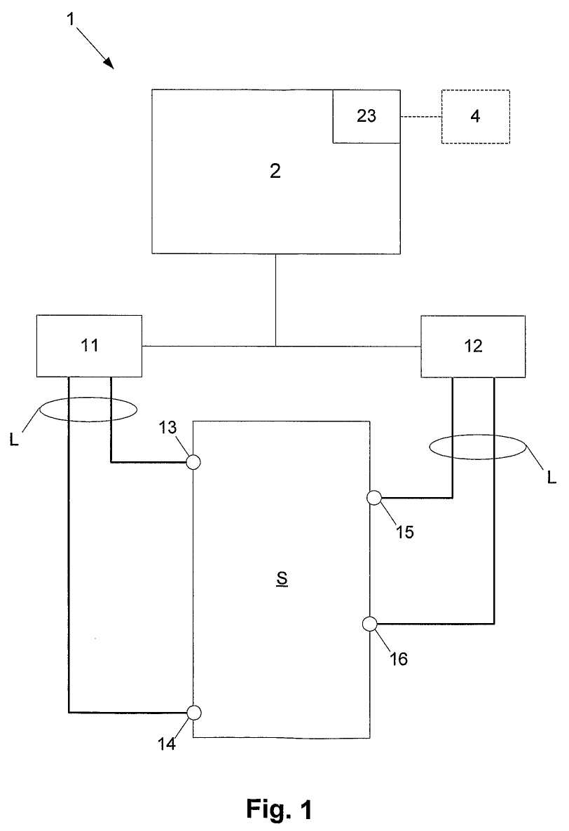

An example of apparatus suitable for performing an analysis of a subject's impedance for the purpose of identifying LVH will now be described with reference to FIG. 1.

As shown the apparatus includes a measuring device 1 including a processing system 2 coupled to a signal generator 11 and a sensor 12. In use the signal generator 11 and the sensor 12 are coupled to respective electrodes 13, 14, 15, 16, provided on a subject S, via leads L, as shown. An optional external interface 23 can be used to couple the measuring device 1 to one or more peripheral devices 4, such as an external database or computer system, barcode scanner, or the like.

In use, the processing system 2 is adapted to generate control signals, which cause the signal generator 11 to generate one or more alternating signals, such as voltage or current signals, which can be applied to a subject S, via the electrodes 13, 14. The sensor 12 then determines the voltage across or current through the subject S using the electrodes 15, 16 and transfers appropriate signals to the processing system 2.

Accordingly, it will be appreciated that the processing system 2 may be any form of processing system which is suitable for generating appropriate control signals and interpreting an indication of measured signals to thereby determine the subject's bioelectrical impedance, and optionally determine other information such as cardiac parameters, or the presence absence or degree of pulmonary oedema.

The processing system 2 may therefore be a suitably programmed computer system, such as a laptop, desktop, PDA, smart phone or the like. Alternatively the processing system 2 may be formed from specialised hardware. Similarly, the I/O device may be of any suitable form such as a touch screen, a keypad and display, or the like.

It will be appreciated that the processing system 2, the signal generator 11 and the sensor 12 may be integrated into a common housing and therefore form an integrated device. Alternatively, the processing system 2 may be connected to the signal generator 11 and the sensor 12 via wired or wireless connections. This allows the processing system 2 to be provided remotely to the signal generator 11 and the sensor 12. Thus, the signal generator 11 and the sensor 12 may be provided in a unit near, or worn by the subject S, whilst the processing system 12 is situated remotely to the subject S.

Once the electrodes are positioned at a suitable location on the subject, an alternating signal is applied to the subject S. This may be performed either by applying an alternating signal at a plurality of frequencies simultaneously, or by applying a number of alternating signals at different frequencies sequentially. The frequency range of the applied signals may also depend on the analysis being performed.

In one example, the applied signal is a frequency rich current from a current source clamped, or otherwise limited, so it does not exceed the maximum allowable subject auxiliary current. However, alternatively, voltage signals may be applied, with a current induced in the subject being measured. The signal can either be constant current, impulse function or a constant voltage signal where the current is measured so it does not exceed the maximum allowable subject auxiliary current.

A potential difference and/or current are measured between an inner pair of electrodes 15, 16. The acquired signal and the measured signal will be a superposition of potentials generated by the human body, such as the ECG, and potentials generated by the applied current.

Optionally the distance between the inner pair of electrodes may be measured and recorded. Similarly, other parameters relating to the subject may be recorded, such as the height, weight, age, sex, health status, any interventions and the date and time on which they occurred. Other information, such as current medication, may also be recorded.

To assist accurate measurement of the impedance, buffer circuits may be placed in connectors that are used to connect the voltage sensing electrodes 15, 16 to the leads L. This ensures accurate sensing of the voltage response of the subject S, and in particular helps eliminate contributions to the measured voltage due to the response of the leads L, and reduces signal loss. This in turn greatly reduces artefacts caused by movement of the leads L.

A further option is for the voltage to be measured differentially, meaning that the sensor used to measure the potential at each electrode 15 only needs to measure half of the potential as compared to a single ended system.

The current measurement system may also have buffers placed in the connectors between the electrodes 13, 14 and the leads L. In one example, current can also be driven or sourced through the subject S symmetrically, which again greatly reduced the parasitic capacitances by halving the common-mode current. Another particular advantage of using a symmetrical system is that the micro-electronics built into the connectors for each electrode 13, 14 also removes parasitic capacitances that arise and change when the subject S, and hence the leads L move.

The acquired signal is demodulated to obtain the impedance of the system at the applied frequencies. One suitable method for demodulation of superposed frequencies is to use a Fast Fourier Transform (FFT) algorithm to transform the time domain data to the frequency domain. This is typically used when the applied current signal is a superposition of applied frequencies. Another technique not requiring windowing of the measured signal is a sliding window FFT.

In the event that the applied current signals are formed from a sweep of different frequencies, then it is more typical to use a processing technique such as multiplying the measured signal with a reference sine wave and cosine wave derived from the signal generator, or with measured sine and cosine waves, and integrating over a whole number of cycles. This process rejects any harmonic responses and significantly reduces random noise.

Other suitable digital and analog demodulation techniques will be known to persons skilled in the field.

Impedance or admittance measurements are determined from the signals at each frequency by comparing the recorded voltage and current signal. The demodulation algorithm will produce an amplitude and phase signal at each frequency.

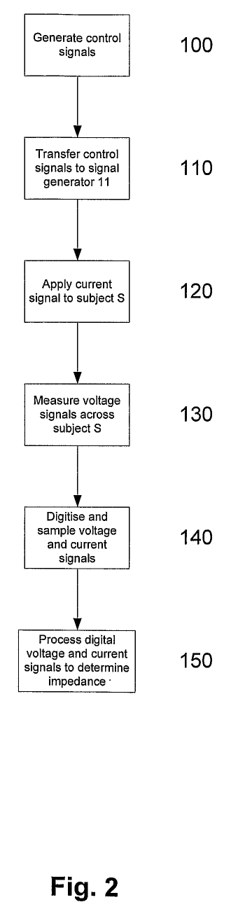

An example of the operation of the apparatus for performing bioimpedance analysis will now be described with reference to FIG. 2.

At step 100, the processing system 2 operates to generate control signals which are provided to the signal generator 11 at step 110, thereby causing the signal generator to apply an alternating current signal to the subject S, at step 120. Typically the signal is applied at each of a number of frequencies fᵢ to allow multiple frequency analysis to be performed.

At step 130 the sensor 12 senses voltage signals across the subject S. At step 140 the measuring device, operates to digitise and sample the voltage and current signals across the subject S, allowing these to be used to determine instantaneous bioimpedance values for the subject S at step 150.

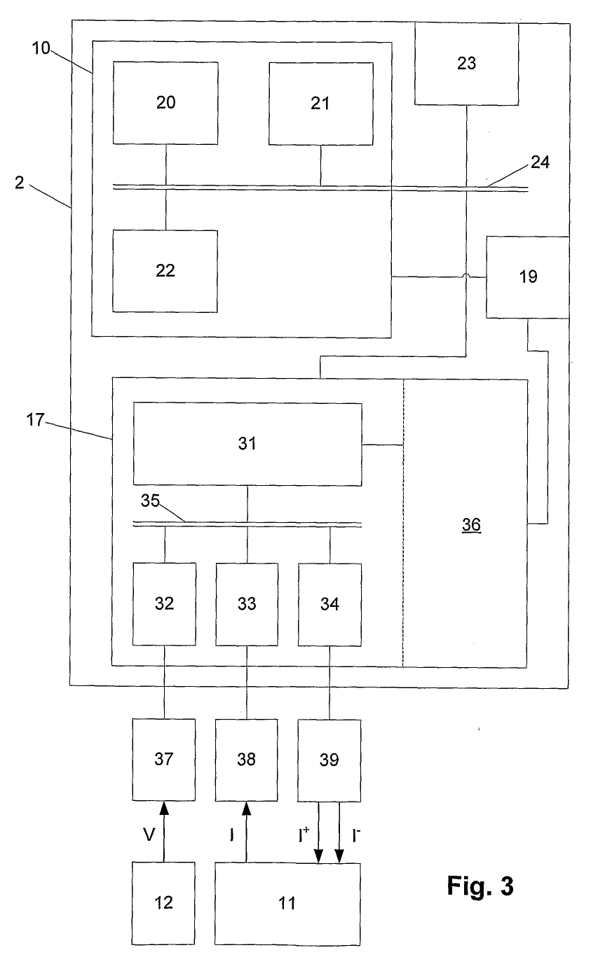

A specific example of the apparatus will now be described in more detail with respect to FIG. 3.

In this example, the processing system 2 includes a first processing system 10 having a processor 20, a memory 21, an input/output (I/O) device 22, and an external interface 23, coupled together via a bus 24. The processing system 2 also includes a second processing system 17, in the form of a processing module. A controller 19, such as a micrologic controller, may also be provided to control activation of the first and second processing systems 10, 17.

In use, the first processing system 10 controls the operation of the second processing system 17 to allow different impedance measurement procedures to be implemented, whilst the second processing system 17 performs specific processing tasks, to thereby reduce processing requirements on the first processing system 10.

Thus, the generation of the control signals, as well as the processing to determine instantaneous impedance values is performed by the second processing system 17, which may therefore be formed from custom hardware, or the like. In one particular example, the second processing system 17 is formed from a Field Programmable Gate Array (FPGA), although any suitable processing module, such as a magnetologic module, may be used.

The operation of the first and second processing systems 10, 17, and the controller 19 is typically controlled using one or more sets of appropriate instructions. These could be in any suitable form, and may therefore include, software, firmware, embedded systems, or the like.

The controller 19 typically operates to detect activation of the measuring device through the use of an on/off switch (not shown). Once the controller detects device activation, the controller 19 executes predefined instructions, which in turn causes activation of the first and second processing systems 10, 17, including controlling the supply of power to the processing systems as required.

The first processing system 10 can then operate to control the instructions, such as the firmware, implemented by the second processing system 17, which in turn alters the operation of the second processing system 17. Additionally, the first processing system 10 can operate to analyse impedance determined by the second processing system 17, to allow biological parameters to be determined. Accordingly, the first processing system 10 may be formed from custom hardware or the like, executing appropriate applications software to allow the processes described in more detail below to be implemented.

It will be appreciated that this division of processing between the first processing system 10, and the second processing system 17, is not essential, but there are a number of benefits that will become apparent from the remaining description.

In this example, the second processing system 17 includes a PCI bridge 31 coupled to programmable module 36 and a bus 35, as shown. The bus 35 is in turn coupled to processing modules 32, 33, 34, which interface with ADCs (Analogue to Digital Converters) 37, 38, and a DAC (Digital to Analogue Converter) 39, respectively.

The programmable module 36 is formed from programmable hardware, the operation of which is controlled using the instructions, which are typically downloaded from the first processing system 10. The firmware that specifies the configuration of hardware 36 may reside in flash memory (not shown), in the memory 21, or may be downloaded from an external source via the external interface 23.

Alternatively, the instructions may be stored within inbuilt memory on the second processing system 17. In this example, the first processing system 10 typically selects firmware for implementation, before causing this to be implemented by the second processing system 17. This may be achieved to allow selective activation of functions encoded within the firmware, and can be performed for example using configuration data, such as a configuration file, or instructions representing applications software or firmware, or the like, as will be described in more detail below.

In either case, this allows the first processing system 10 to be used to control operation of the second processing system 17 to allow predetermined current sequences to be applied to the subject S. Thus, for example, different firmware would be utilised if the current signal is to be used to analyse the impedance at a number of frequencies simultaneously, for example, by using a current signal formed from a number of superposed frequencies, as compared to the use of current signals applied at different frequencies sequentially.

This allows a range of different current sequences can be applied to the subject by making an appropriate measurement type selection. Once this has been performed, the FPGA operates to generate a sequence of appropriate control signals I+, I−, which are applied to the subject S. The voltage induced across the subject being sensed using the sensor 12, allowing the impedance values to be determined and analysed by the second processing system 17.

Using the second processing system 17 allows the majority of processing to be performed using custom configured hardware. This has a number of benefits.

Firstly, the use of a second processing system 17 allows the custom hardware configuration to be adapted through the use of appropriate firmware. This in turn allows a single measuring device to be used to perform a range of different types of analysis.

Secondly, this vastly reduces the processing requirements on the first processing system 10. This in turn allows the first processing system 10 to be implemented using relatively straightforward hardware, whilst still allowing the measuring device to perform sufficient analysis to provide interpretation of the impedance. This can include for example generating a “Wessel” plot, using the impedance values to determine parameters relating to cardiac function.

Thirdly, this allows the measuring device 1 to be updated. Thus for example, if an improved analysis algorithm is created, or an improved current sequence determined for a specific impedance measurement type, the measuring device can be updated by downloading new firmware via flash memory (not shown) or the external interface 23.

It will be appreciated that in the above examples, the processing is performed partially by the second processing system 17, and partially by the first processing system 10. However, it is also possible for processing to be performed by a single element, such as an FPGA, or a more generalised processing system.

As the FPGA is a customisable processing system, it tends to be more efficient in operation than a more generic processing system. As a result, if an FPGA alone is used, it is generally possible to use a reduced overall amount of processing, allowing for a reduction in power consumption and size. However, the degree of flexibility, and in particular, the range of processing and analysis of the impedance which can be performed is limited.

Conversely, if only a generic processing system is used, the flexibility is enhanced at the expense of a decrease in efficiency, and a consequent increase in size and power consumption.

Accordingly, the above described example strikes a balance, providing custom processing in the form of an FPGA to perform partial processing. This can allow for example, the impedance values to be determined. Subsequent analysis, which generally requires a greater degree of flexibility can then be implemented with the generic processing system.

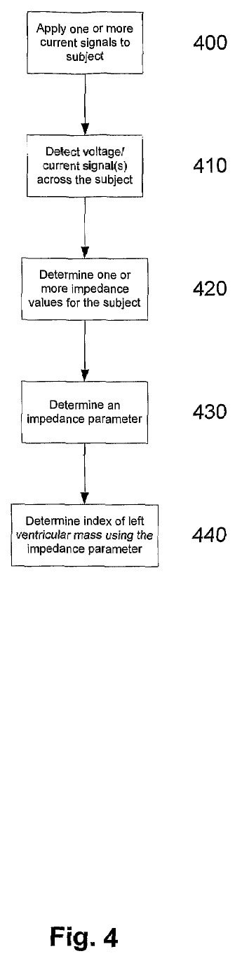

An example of the process for performing impedance measurements utilising the apparatus to FIGS. 1 or 3 to provide an index of LVM will now be described with reference to FIG. 4.

At step 400 one or more current signals are applied to a subject with the measuring device 1 being used to detect voltage/current signals across the subject step 410. The current and voltage signals are then used to determine one or more impedance values for the subject at step 420, with these being used to determine an impedance parameter at step 430. The impedance parameter can then be used to determine an index of LVM at step 440, which may in turn be used in the assessment of the presence, absence or degree of LVH.

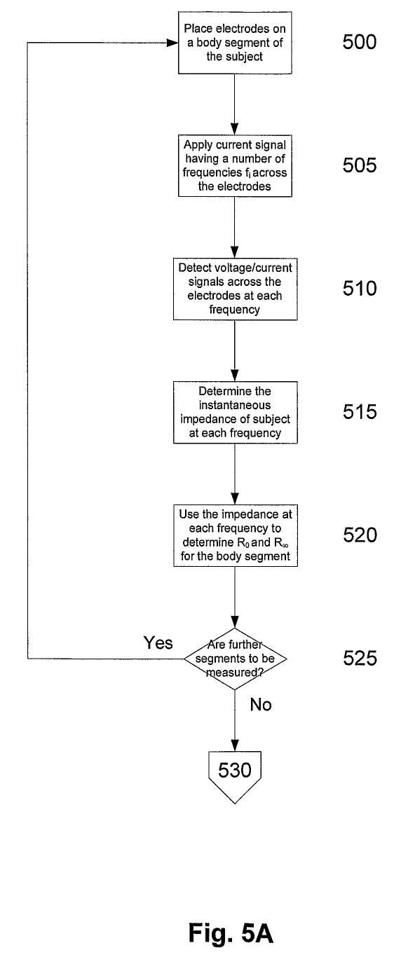

A specific example of the manner in which this is achieved for specific electrode placements will now be described with reference to FIGS. 5A and 5B.

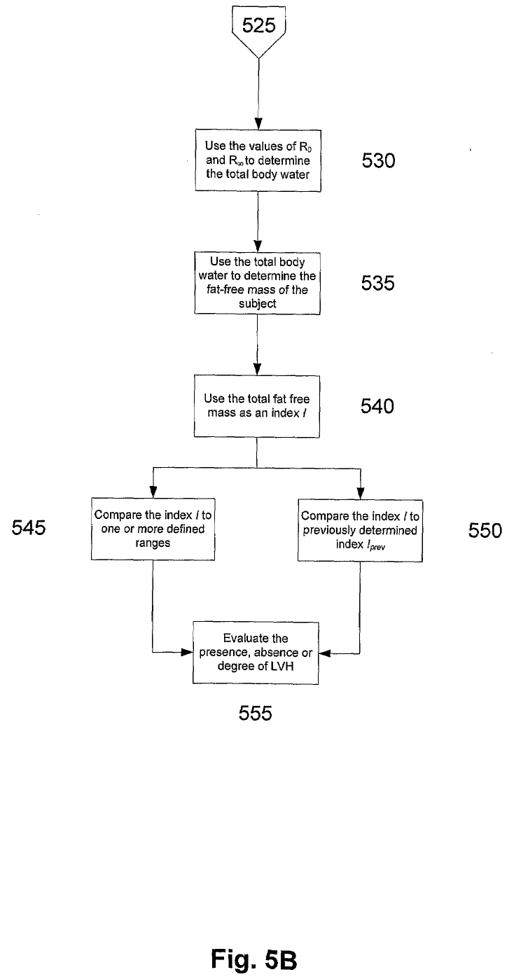

At step 500 electrodes are placed on a body segment of the subject. The electrode configurations used will vary depending on the type of apparatus available, the circumstances in which the system is used, or the like. Example configurations are shown in FIGS. 6A to 6D.

In this regard, the electrode configurations shown in FIGS. 6A to 6D involve positioning electrodes on the limbs of the subject S, with the particular electrode placement allowing the impedance of different body segments to be measured.

In the examples of FIGS. 6A and 6B, the configuration allows the impedance of the entire subject to be determined, whereas the configurations shown in FIGS. 6C and 6D allow the right arm 631 and the right leg 633 to be measured respectively.

In general, when such an electrode arrangement is used, it is typical to provide electrodes in each possible electrode placement position, with leads being connected selectively to the electrodes as required. This will be described in more detail below.

It will be appreciated that this configuration uses the theory of equal potentials, allowing the electrode positions to provide reproducible results for impedance measurements. For example when current is injected between electrodes 13 and 14 in FIG. 6C, the electrode 16 could be placed anywhere along the left arm 632, since the whole arm is at an equal potential.

This is advantageous as it greatly reduces the variations in measurements caused by poor placement of the electrodes by the operator. It also greatly reduces the number of electrodes required to perform segmental body measurements, as well as allowing the limited connections shown to be used to measure each of limbs separately.

At step 505 current signals having a number of frequencies fᵢ are applied across the electrodes with voltage and current signals across the electrodes being detected at each frequency at step 510. At step 515 the processing system 10 operates to determine the instantaneous impedance of the body segment at each frequency, using these to determine R₀ and R_{∞} for the body segment at step 520.

This can be achieved in a number of manners as will now be described.

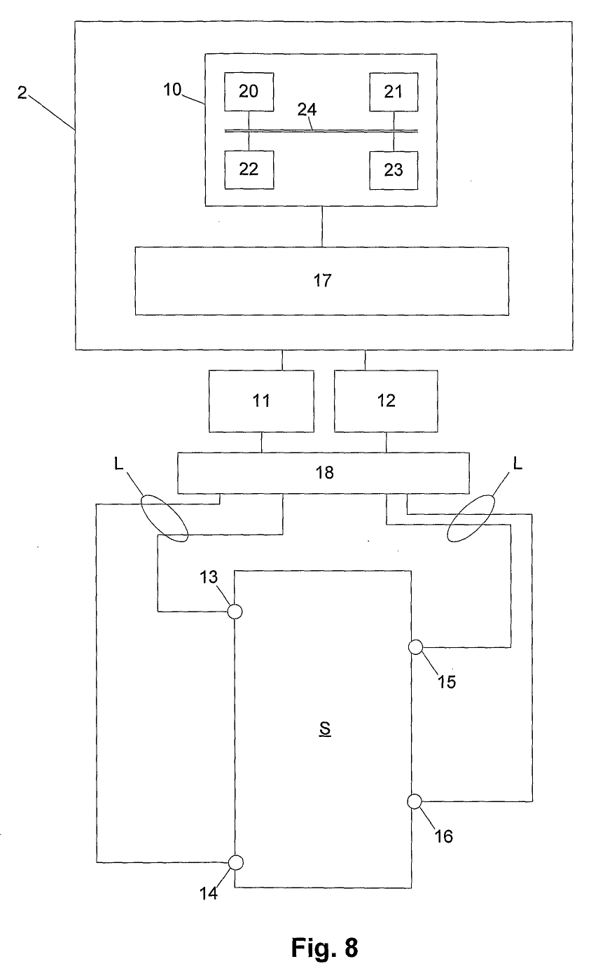

In this regard, FIG. 9 is an example of an equivalent circuit that effectively models the electrical behaviour of biological tissue. The equivalent circuit has two branches that represent current flow through extracellular fluid and intracellular fluid. The extracellular component of biological impedance is represented by Rₑ and the intracellular component is represented by Rᵢ. Capacitance associated with the cell membrane is represented by C.

The relative magnitudes of the extracellular and intracellular components of impedance of an alternating current (AC) are frequency dependent. At zero frequency the capacitor acts as a perfect insulator and all current flows through the extracellular fluid, hence the resistance at zero frequency, R₀, equals Rₑ. At infinite frequency the capacitor acts as a perfect conductor and the current passes through the parallel resistive combination. The resistance at infinite frequency is given by R₂₈=RᵢRₑ/(Rᵢ+Rₑ).

Accordingly, the impedance of the equivalent circuit of FIG. 9 at an angular frequency ω, where ω=2π*frequency, is given by:

$$Z = {R}_{∞} + \frac{{R}_{0} - {R}_{∞}}{1 + ( jωτ )} \qquad ( 1 )$$

where:
   R_{∞}=impedance at infinite applied frequency=RᵢRₑ/(Rᵢ+Rₑ),
   R₀=impedance at zero applied frequency=Rₑ and,
τ is the time constant of the capacitive circuit.

However, the above represents an idealised situation which does not take into account the fact that the cell membrane is an imperfect capacitor. Taking this into account leads to a modified model in which:

$$Z = {R}_{∞} + \frac{{R}_{0} - {R}_{∞}}{1 + {( jωτ )}^{( 1 - α )}} \qquad ( 2 )$$

where α has a value between 0 and 1 and can be thought of as an indicator of the deviation of a real system from the ideal model.

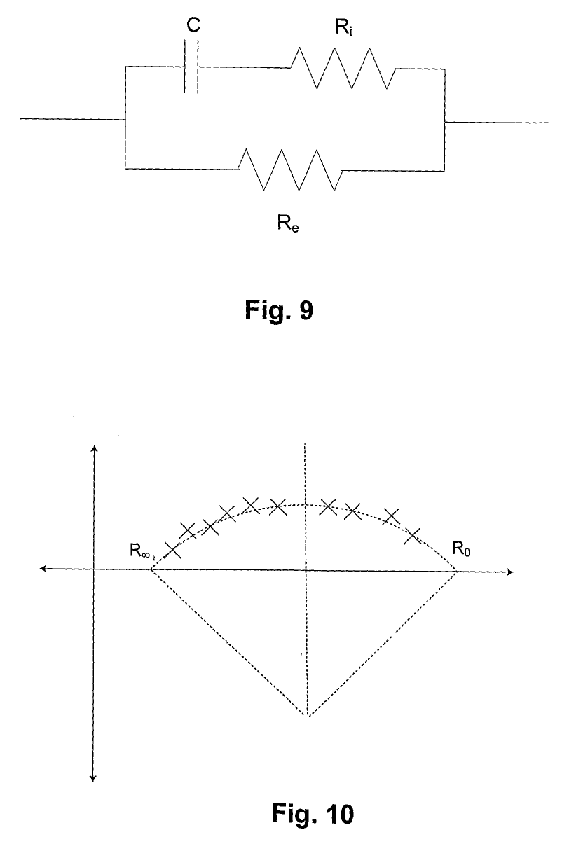

The value of the impedance parameters R₀ and R_{∞} may be determined in any one of a number of manners such as by:
solving simultaneous equations based on the impedance values determined at different frequencies;
using iterative mathematical techniques;
extrapolation from a “Wessel plot” similar to that shown in FIG. 10;
performing a function fitting technique, such as the use of a polynomial function.

The above described equivalent circuit models the resistivity as a constant value and does not therefore accurately reflect the impedance response of a subject or other relaxation effects. To more successfully model the electrical conductivity of a human, an improved CPE based may alternatively be used.

In any event, it will be appreciated that any suitable technique for determination of the parameter values R₀ and R_{∞} may be used.

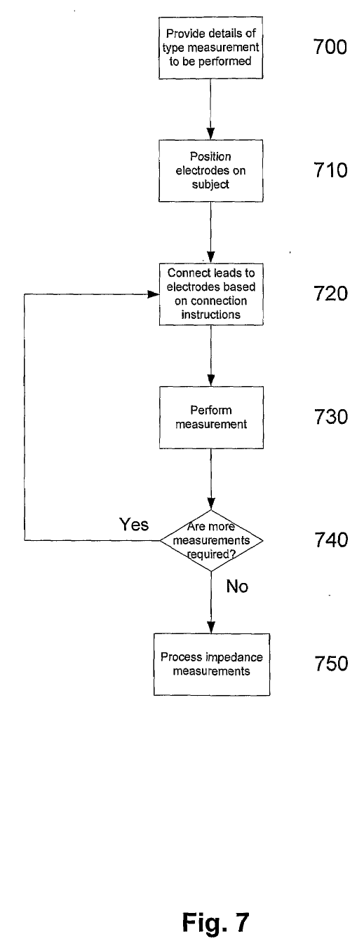

This may be performed for a single body segment, such as the entire body, using the electrode arrangements shown in FIGS. 6A or 6B. Alternatively, the may be performed on a number of smaller body segments, such as the limbs, and/or thoracic cavity separately, using for example the electrode configurations shown in FIGS. 6C to 6D. A combination of the two approaches may also be used. The electrode configurations can also be selected automatically using a multi-channel system, such as that described below with respect to FIG. 8.

If further body segments are to be measured at step 525 the process returns to step 500 allowing a suitable electrode placement to be determined as required.

Otherwise, once all body segments have been determined, the derived values of R₀ and R_{∞} are used to determined the total body water for the subject at step 530. This can be achieved using equations formulated from Hanai's theory. In particular, this indicates that the total body water is given by:

TBW=ecf +icf (3)
where:
   TBW=total body water
   ecf=volume of extracellular fluid
   icf=volume of intracellular fluid

In this regard, the volumes of extracellular and intracellular water can be derived from the values R₀, R_{∞}, as these depend on the values of the extracellular and intracellular resistance, as discussed above.

An example of the process for determining ecf based on the method of Van Loan et al (“Use of bioelectrical impedance spectroscopy (BIS) to measure fluid changes during pregnancy”—J. Appl Physiol. 78:1037-1042, 1995), modified to take into account body proportion using the formulae of De Lorenzo et al (“Predicting body cell mass with bioimpedance by using theoretical methods: a technological review”.—J. Appl. Physiol. 82(5):1542-1558, 1997).

In particular, the extracellular fluid is given by:

$$ecf = \frac{\sqrt[3]{\frac{{p}^{2} {ρ}_{ecw}^{2}}{d}} \sqrt[3]{\frac{{h}^{4} w}{{R}_{0}^{2}}}}{100} \qquad ( 4 )$$

where:
   h=subject's height
   p=subjects body proportion,
   d=subject's body density,
   ρₑ=subject's extracellular resistivity (sex dependent)

The icf is then given by:

$${( 1 + \frac{icf}{ecf} )}^{\frac{5}{2}} = ( \frac{{R}_{e} + {R}_{i}}{R} ) ( 1 + \frac{{ρ}_{i}}{{ρ}_{e}} \frac{icf}{ecf} ) \qquad ( 5 )$$

where: ρᵢ=subject's intracellular resistivity

This can be solved by expanding into the form shown in equation (6) and solving iteratively by using various values of x between 0 and 5, until the result is approximately zero (within 0.00001).

$${x}^{5} + 5 {x}^{4} + 10 {x}^{3} + ( 10 - {( \frac{{R}_{0}}{{R}_{∞}} )}^{2} {( \frac{{ρ}_{i}}{{ρ}_{e}} )}^{2} ) {x}^{2} + ( 5 - 2 {( \frac{{R}_{0}}{{R}_{∞}} )}^{2} ( \frac{{ρ}_{i}}{{ρ}_{e}} ) ) x + 1 - {( \frac{{R}_{0}}{{R}_{∞}} )}^{2} = 0 \qquad ( 6 )$$

where:

$$x = \frac{icf}{ecf}$$

The icf can then be calculated from x and ecf determined using (4) above.

At step 535, the processing system 10 uses the total body water to determine the fat free mass FFM of the subject. Again this may be achieved in any one of a number of manners such as using the “Hanai” theory, in which the FFM is given by:

FFM=TBW/0.732 (7)
where: 0.732 is the default hydration constant

At step 540 the total fat free mass can be used to index left ventricle mass, as has previously been performed with respect to DEXA analysis.

This can be achieved for example by using the LVM determined from measurement, such as echocardiography. The index I is then given by:

I=LVM/FFM (8)

It will be appreciated that once the LVM has been indexed, the index can be used for determining whether the subject suffers from LVH. This is typically achieved by comparing the index I to a reference to determine if the subject suffers from LVH. The comparison may be performed automatically by the first processing system 10. Additionally or alternatively, this may involve having the processing system 10 display the index, fat free mass, or left ventricular mass as estimated from the fat free mass, and a corresponding reference, to allow a visual comparison by an operator.

At step 545, the reference can be based on predetermined normal ranges of expected index values, fat-free mass values, or left ventricular mass values, as estimated from the fat-free mass. This can be derived, for example, from a study of a number of other individuals, and may therefore depend on other factors relating to the subject, such as subject parameters including but not limited to the age, weight, sex, height and ethnicity of the subject. In this instance, the processing system 10 could be provided with respective information relating to the subject, with this being used to access a predetermined range stored in the memory 21. If the measured LVM falls outside the predefined range, this can indicate the presence, absence or degree of LVH.

Alternatively, or additionally, at step 550, a longitudinal analysis is performed, in which a current value for the index I can be compared to previously determined index values Iₚᵣₑᵥ for the subject to determine if there has been a change in the LVM index and hence LVH status.

It will be appreciated that these techniques may be used in conjunction with one another for more accurate assessment on the development, and in particular, the presence, absence or degree of LVH within the subject at step 555.

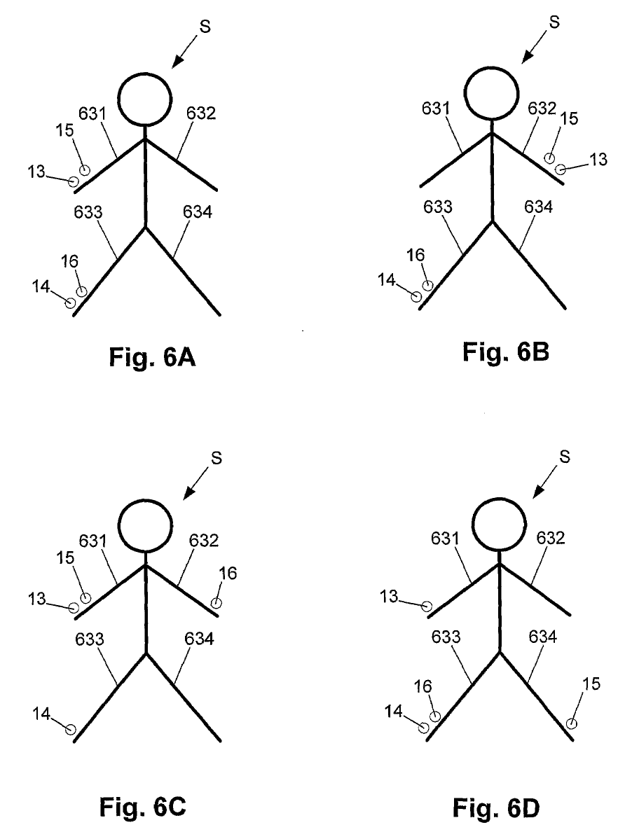

In the above described process, if a number of different body segments are measured, a number of different electrode placements may be required. An explanation of a process for electrode replacement will now be described with reference to FIG. 7.

At step 700 an operator of the apparatus provides details of a type of impedance measurement to be performed to the measuring device. Thus, for example, the operator will indicate that the LVM is to be determined as well as indicating whether or not electrodes will be provided on the body as shown in FIG. 6A to FIG. 6D.

At step 710 the operator positions electrodes on the subject, and this typically involves placing electrode pads at each position where electrodes will be required during the measurement process. Following this the operator connects leads to the electrode pads based on connection instructions provided by the measuring device at step 720.

It will therefore be appreciated that this may be achieved in a number of ways and that typically, this involves having the measuring device 1 present a list of the available measurement types and allow the user to select the measurement type of interest. This can then be used to access a profile specifying the required electrode arrangement, which is then displayed to the user, allowing the user to correctly connect the electrodes.

At step 730 the measuring device 1 will operate to perform impedance measurements by generating an appropriate current sequence and applying this to the subject via the electrodes 13, 14.

At step 740 the measuring device 1 determines if further impedance measurements are required and if so the process returns to step 720 to allow the operator to connect leads to different ones of the electrodes as required. This process is repeated until sufficient impedance measurements have been collected to perform the required analysis.

At this stage, the process moves on to step 750 with the measuring device operating to process the impedance measurements and provide an indication of required information to the operator, as described above.

Accordingly, this provides instruction to the operator allowing the operator to ensure accurate electrode placement, thereby further enhancing the accuracy of the measurement process.

Alternatively however an automated system may be used in which electrodes are positioned at each of the potential measurement positions, with leads being connected to each of the electrodes. This allow the measuring device to automatically apply current to the appropriate electrodes.

This may be achieved utilising apparatus shown in FIG. 8 in which the measuring device 1 includes a switching arrangement. In this example, the measuring device 1 includes a switching device 18, such as a multiplexer, for connecting the signal generator 11 and the sensor 12 to the leads L. This allows the measuring device 1 to control which of the leads L are connected to the signal generator 11 and the sensor 12.

In this example, a single set of leads and connections is shown. This arrangement can be used in a number of ways. For example, by identifying the electrodes 13, 14, 15, 16 to which the measuring device 1 is connected, this can be used to control to which of the leads L signals are applied, and via which leads signals can be measured. This can be achieved either by having the user provide an appropriate indication via the input device 22, or by having the measuring device 1 automatically detect electrode identifiers.

Alternatively, however the arrangement may be used with multiple leads and electrodes to provide multi-channel functionality.

In this example, the electrodes 13, 14, 15, 16 are provided on the subject at respective locations, such as in each of the possible electrode locations shown in FIGS. 6A to 6D. The multiplexing of signals can be controlled by the processing system 10, or the FPGA 17 if present, thereby allowing the measuring device 1 to apply a current to selected ones of the electrodes in turn, measuring the resulting potentials at corresponding ones of the remaining electrodes automatically.

In any event, it is apparent that the above described methodology allows determination of fat-free mass, and hence determination of an LVM index, which can be used in assessing the presence, absence or degree of LVH. This avoids the need for complex apparatus such as DEXA systems.

Persons skilled in the art will appreciate that numerous variations and modifications will become apparent. All such variations and modifications which become apparent to persons skilled in the art, should be considered to fall within the spirit and scope that the invention broadly appearing before described.

Thus, for example, it will be appreciated that features from different examples above may be used interchangeably where appropriate. Furthermore, whilst the above examples have focussed on a subject such as a human, it will be appreciated that the measuring device and techniques described above can be used with any animal, including but not limited to, primates, livestock, performance animals, such race horses, or the like.

It will also be appreciated above described techniques, may be implemented using devices that do not utilise the separate first processing system 10 and second processing system 17, but rather use a single processing system 2, or use some other internal configuration.

1-19. (canceled)

20. A method of determining an index indicative of the presence, absence or degree of left ventricular hypertrophy in a subject, the method including, in a processing system:
(a) determining a measured impedance value for at least one body segment;
(b) for each body segment, and using the measured impedance values, determining at least one impedance parameter value;
(c) using each determined impedance value to determine a fat-free mass for the subject; and,
(d) determining the index at least in part using the fat-free mass.

21. A method according to claim 20, wherein the method includes, in the processing system determining the index using the fat-free mass and an indication of a measured left ventricular mass.

22. A method according to claim 20, wherein the method includes, in the processing system:
(a) comparing the index to a reference; and,
(b) determining the presence, absence or degree of LVH using the results of the comparison.

23. A method according to claim 22, wherein the reference includes at least one of:
(a) a predetermined threshold;
(b) a tolerance determined from a normal population;
(c) a predetermined range; and,
(d) an index previously determined for the subject.

24. A method according to claim 20, wherein the method includes, in the processing system, displaying at least one of:
a) a fat free mass;
b) a determined index;
c) a ventricular mass;
d) normal ranges for the index; and,
e) normal ranges for fat free mass; and,
f) normal ranges left ventricular mass.

25. A method according to claim 24, wherein the method includes determining the ranges in accordance with subject parameters.

26. A method according to claim 20 wherein the method includes, in the processing system:
(a) determining a plurality of measured impedance values for each body segment, each measured impedance value being measured at a corresponding measurement frequency; and,
(b) determining the impedance parameters based on the plurality of measured impedance values.

27. A method according to claim 20, wherein the parameter values include R₀ and R₀₀, wherein:
R₀ is the resistance at zero frequency; and,
R_{∞} is the resistance at infinite frequency.

28. A method according to claim 25, wherein the method includes, in the processing system, determining the parameter values using the equation:
$$Z = {R}_{∞} + \frac{{R}_{0} - {R}_{∞}}{1 + {( jωτ )}^{( 1 - α )}}$$
where:
Z is the measured impedance at angular frequency ω,
τ is a time constant, and
α has a value between 0 and 1.

29. A method according to claim 26, wherein the method includes, in the processing system:
(a) determining the impedance of each body segment at four discrete frequencies; and,
(b) determining values for the parameters by solving the equation using four simultaneous equations.

30. A method according to claim 26, wherein the method includes, in the processing system, determining the parameter values by:
(a) determining an impedance locus using the measured impedance values; and,
(b) using the impedance locus to determine the parameter values.

31. A method according to claim 20, wherein the method includes, in the processing system:
(a) causing one or more electrical signals to be applied to the subject using a first set of electrodes, the one or more electrical signals having a plurality of frequencies;
(b) determining an indication of electrical signals measured across a second set of electrodes applied to the subject in response to the applied one or more signals;
(c) determining from the indication and the one or more applied signals, an instantaneous impedance value at each of the plurality of frequencies; and,
(d) determining the index using the instantaneous impedance values.

32. A method according to claim 20, wherein the method includes, in the processing system:
(a) determining at least one impedance measurement to be performed;
(b) determining at least one electrode arrangement associated with the determined impedance measurement;
(c) displaying a representation indicative of the electrode arrangement; and,
(d) causing the impedance measurement to be performed once the electrodes have been arranged in accordance with the displayed representation.

33. A method according to claim 20, wherein the method includes, in the computer system, displaying an indication of at least one of:
(a) the parameter values;
(b) the fat-free mass; and,
(c) an indication of the at least one of the presence, absence or degree of LVH.

34. Apparatus for determining an index indicative of the presence, absence or degree of left ventricular hypertrophy in a subject, the apparatus includes a processing system for:
(a) determining a measured impedance value for at least one body segment;
(b) for each body segment, and using the measured impedance values, determining at least one impedance parameter value; and,
(c) using each determined impedance value to determine a fat-free mass for the subject; and,
(d) determining at least in part the index using the fat-free mass.

35. Apparatus according to claim 34, wherein the apparatus includes:
(a) a current supply for generating an alternating current at each of a plurality of frequencies;
(b) at least two supply electrodes for applying the generated alternating current to a subject;
(c) at least two measurement electrodes for detecting a voltage across the subject; and,
(d) a sensor coupled to the measurement electrodes for determining the voltage, the sensor being coupled to the processing system to thereby allow the processing system to determine the measured impedances.

36. Apparatus according to claim 34, wherein the processing system is for performing the method of claim 20.

37. A method of diagnosing the presence, absence or degree of left ventricular hypertrophy in a subject, the method including, in a processing system:
(a) determining a measured impedance value for at least one body segment;
(b) for each body segment, and using the measured impedance values, determining at least one impedance parameter value;
(c) using each determined impedance value to determine a fat-free mass for the subject; and,
(d) determining an index at least in part using the fat-free mass, the index being indicative of the presence, absence or degree of left ventricular hypertrophy.

38. The method of claim 20 further comprising:
(a) determining a measured impedance value for at least one body segment;
(b) for each body segment, and using the measured impedance values, determining at least one impedance parameter value;
(c) using each determined impedance value to determine a fat-free mass for the subject; and,
(d) determining an index at least in part using the fat-free mass, the index being indicative of the presence, absence or degree of left ventricular hypertrophy.
